(19) United States
(12) Patent Application Publication
(10) Pub. No.: US 2025/0296983 A1
(43) Pub. Date: Sep. 25, 2025

(54) IgY Antibody Compositions and Methods for Treating Mammal Species

(71) Applicant: Mammal Labs LLC, San Jose, CA (US)

(72) Inventors: Vinicius Pereira de Lima Junior, Gainesville, FL (US); Robert A. Burtzlaff, Coos Bay, OR (US); David R. Stevens, Los Gatos, CA (US)

(21) Appl. No.: 19/085,670

(22) Filed: Mar. 20, 2025

Related U.S. Application Data
(60) Provisional application No. 63/568,179, filed on Mar. 21, 2024.

(51) Int. Cl. C07K 16/08; A61K 9/50; A61K 35/20; A61K 39/00

(52) U.S. Cl. CPC C07K 16/081; A61K 9/50; A61K 35/20; A61K 2039/505; A61K 2039/54; C07K 2317/11; C07K 2317/23

(57) ABSTRACT

A composition and method for therapeutic or prophylactic treatment of mammals includes a mixture of IgY antibodies derived from one or more eggs laid by one or more avian species, wherein each of the one or more avian species have been vaccinated with one or more of the plurality of antigens. The mixture of IgY antibodies can be combined with a protective material that includes colostrum. In some embodiments the composition can also include IgA and IgM antibodies.

### RELATED APPLICATION

This application claims the benefit of U.S. Provisional Application Ser. No. 63/568,179, filed Mar. 21, 2024, and entitled IgY Antibody Compositions and Methods for Treating Avian Species, which is hereby incorporated herein by reference.

### FIELD OF INVENTION

The invention provides compositions and methods for treatment of mammal species using compositions that include IgY antibodies.

### BACKGROUND OF THE DISCLOSURE

Antibodies, both naturally occurring and their synthetic analogues, are known therapeutic agents for mammals. Antibodies bind a portion of the antigen (the antigenic determinant or epitope) with an antibody combining site or antigen binding site. Antibodies are capable of high degrees of specificity enabling targeted application to specific pathogens or toxins.

IgY antibodies from avian eggs have been shown to be effective against pathogens and toxins. Unfortunately, orally delivered antibody therapeutic effectiveness is diminished by passage through the stomach and exposure to gastric acid and digestion enzymes. Improved IgY delivery methods for treatment of mammal or other animal species are needed.

### SUMMARY OF THE INVENTION

A composition and method for therapeutic or prophylactic treatment of mammals includes a mixture of IgY antibodies derived from one or more eggs laid by one or more avian species. In some embodiments, each of the one or more avian species have been vaccinated with one or more of the plurality of antigens. Alternatively, antibodies naturally present in eggs can be used. A protective material such as colostrum that slows degradation of the mixture of IgY and optional antibodies in an animal gut can be used in conjunction with an encapsulating material to hold together the mixture of IgY antibodies and protective material.

In another embodiment, an intravenously deliverable composition for therapeutic or prophylactic treatment of animals includes a mixture of IgY antibodies derived from one or more eggs laid by one or more avian species, wherein each of the one or more avian species have been vaccinated with one or more of the plurality of antigens. Also provided is mixture of antibodies (including but not limited to IgY, IgA, and IgM) and a protective material such as colostrum that slows degradation of the mixture of IgY and other antibodies and is soluble in saline solution.

### BRIEF DESCRIPTION OF THE DRAWINGS

In order that the advantages of the invention will be readily understood, a more particular description of the invention briefly described above will be rendered by reference to specific embodiments illustrated in the appended drawing. Understanding that this drawing depicts only a typical embodiment of the invention and is not therefore to be considered limiting of its scope, the invention will be described and explained with additional specificity and detail through use of the accompanying drawing.

FIG. 1 is a schematic drawing of a technique for producing the disclosed composition; and

FIG. 2 illustrates a representative treatment or prophylactic method.

### DETAILED DESCRIPTION OF THE DISCLOSURE

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “organism” means a form which may have a single cell or multiple cells, and which include bacteria, viruses, parasites, and fungi.

As used herein, “pathogen” means an organism which causes disease or untoward effects in a host and which include bacteria, viruses, and parasites.

As used herein, “toxin” means poison or substance produced by a pathogen and that causes disease or untoward effects in a host.

While this invention is susceptible of embodiment in many different forms, there is shown in the drawings, which will herein be described in detail, specific embodiments with the understanding that the present disclosure is to be considered as an exemplification of the principals of the invention and is not intended to limit the invention to the illustrated embodiments.

Disclosed is a prophylactic or therapeutic composition that includes IgY antibodies derived from eggs laid by chickens or other avian species (e.g. egg-laying hens). These avian species can be immunized against one or more pathogens toxins.

In those embodiments where chickens are used to produce the eggs, the chickens may be any domestic bird of the subspecies Gallus gallus domesticus. Examples include, but are not limited to, the following breeds of Gallus domesticus: Rhode Island Red, Leghorn, Australorp, Lohmann Brown Classic, Sussex, Golden Comet, Marans, Plymouth Rock, Barnevelder, Buff Orpington, Ameraucana, La Brese, and Hamburg. These examples are breeds of chickens which are known to be prolific egg producers. However, other breeds of chickens and other hens of avian species are within the scope of this disclosure. For example, egg laying avians can include ducks, turkeys, geese, or ostriches.

In one embodiment, prior to egg collection, the hens may be immunized with a vaccine comprising at least one antigen which initiates production of antibodies directed against one or more pathogens or toxins. Alternatively or in addition, adjuvants can be added to increase production of naturally present or vaccine induced antibodies. The vaccine may be produced by any method known in the art. Examples include attenuated live vaccines, modified live vaccines, chemically altered vaccines, killed vaccines, toxoid vaccines, DNA vaccines, subunit vaccines, recombinant vaccines, polysaccharide vaccines, and conjugate vaccines. The vaccines may be directed against viruses, bacterial pathogens, parasites, yeasts, or molds. The vaccines may also be directed against toxins, poisons or adhesins produced by pathogens. In some embodiments, the vaccines may include one or more adjuvants which enhance the immunogenicity of the vaccine.

In some embodiments, the avian species may be immunized with vaccines which include a live, wild-type pathogen. Vaccines are typically created using pathogens which have been rendered less virulent (by modifying or killing it) and unlikely to result in clinical illness in the organism receiving the vaccine.

In some embodiments, each member of an avian species may be immunized with a single pathogen. For example, multiple chicken hens may each be immunized with a different single antigen and the eggs from each hen may be combined to result in a product which includes IgY antibodies against different antigens. In other embodiments, a single hen may be immunized with two or more antigens resulting in IgY antibodies against the different antigens present in the same egg.

In some embodiments, pathogens against which the avian species may be immunized include one or more of a species of the genus Clostridium, a species of the genus Yersinia, a species of the genus Brachyspira, a species of the genus Campylobacter, Escherichia coli, Helicobacter spp., a species of the genus Salmonella, a species of the genus Leptospira, Providencia alcalifaciens, Mycobacterium paratuberculosis, Lawsonia intracellularis, bovine viral diarrhea virus, infectious bovine rhinotracheitis virus, coronavirus, rotavirus, parvovirus, paramyxovirus, hepatitis virus, feline leukemia virus, feline immunodeficiency virus, feline panleukopenia virus, astrovirus, transmissible gastroenteritis, Giardia, Coccidia, Entamoeba histolytica, Cryptosporidium, roundworm, whipworm, spirochetes, yeasts, and molds.

In some embodiments, birds can be immunized against various pathogens often associated with illness in mammal species. These can include but are not limited to dogs, cats, cattle, horses, swine, sheep, goats, camels, rabbits, guinea pigs, chinchillas, and other animals that are within the scope of this disclosure. In some embodiments, the animals may be non-neonate animals.

In an example, calves from bovine species often suffer from diarrhea caused by one or more of a variety of infectious pathogens. Calf scours represents a significant loss to cow-calf producers. Examples of infectious pathogens which may cause calf scours include E. coli, Salmonella spp., Clostridium perfringens, rotavirus, corona virus, bovine viral diarrhea (BVD) virus, infectious bovine rhinotracheitis (IBR) virus, cryptosporidium, Coccidia, yeasts, and molds.

Similarly, the young offspring of equine species often suffer from pathogen-induced diarrhea. These pathogens including C. perfringens, C. difficile, Salmonella spp., E. coli, Cryptosporidium parvum, rotavirus, coronavirus, and Aeromonas hydrophilia.

Swine are also prone to diarrhea caused by pathogens. Examples of pathogens which may cause diarrhea in swine include Brachyspira pilosicoli, atypical Brachyspira, Brachyspira hyodysenteriae, Lawsonia intracellularis, Salmonella spp., Salmonella typhimurium, Salmonella choleraesuis, Yersinia pseudotuberculosis, Escherichia coli, Clostridium perfringens, Cryptosporidium spp., Giardia spp., transmissible gastroenteritis (TGE) virus, African swine fever virus (genotypes I-XXIII), and whipworm.

Sheep and goats are known to suffer from diarrhea-causing illnesses as a result of pathogens. These pathogens include coccidia, species of the genus Yersinia (including Yersinia pseudotuberculosis and Yersinia enterocolitica), species of the genus Salmonella, Escherichia coli, Cryptosporidium spp., Clostridium perfringens, Mycobacterium paratuberculosis, rotavirus, and coronavirus. Worms (GI helminths) are also a common cause of diarrhea in sheep and goats.

The disclosed composition and methods of use may be applied to pets or livestock species discussed above and others suffering from diarrhea or other infectious gastrointestinal diseases. In addition to treating or preventing diarrhea, the disclosed embodiments may be used to produce compositions which treat or prevent other gastrointestinal diseases and symptoms including colitis, constipation, bloating, gastritis, gastrointestinal ulcers, gastrointestinal tumors, hemorrhagic gastritis, inflammatory bowel disease, and malabsorption. Rather than targeting diarrhea-causing organisms and toxins, the antibodies will be created to adhere to organisms and toxins which cause one of the listed gastrointestinal diseases or symptoms.

In some subjects, the gastrointestinal discomfort may be the result of an imbalance in the subject's gastrointestinal biome. Opportunistic microbial organisms may take over the gastrointestinal biome at the expense of other microbial species. This may be the result of a variety of insults to the gastrointestinal tract including improper diet, antibiotics, and pathogen ingestion. Antibodies which adhere to the opportunistic microbial organisms may be produced as disclosed herein and mixed with a protective protein matrix. The mixture may be given orally, and the antibodies may adhere to and neutralized or facilitate destruction of the opportunistic microbes. When given in the proper titer, the composition may restore balance to the subject's gastrointestinal biome.

In some embodiments, cross-reactive antibodies may also be used to produce compositions to treat infections outside of the gastrointestinal tract. External infections or those within bodily orifices and tissues which may be accessed with minimally invasive procedures and can be treated with cross-reactive antibody mixtures as disclosed herein. These infections may be those occurring in parts of the body including the ear, throat, skin, and urinary bladder. A protective protein matrix may be mixed with the antibodies to protect them from degradation in the environment to be treated. The cross-reactivity of the antibodies negates the need to identify the precise species of the infecting organism.

This treatment confers passive immunity to patients. The nature of the treatment makes the associated risk factors comparable to that of eating food from the source where the antibodies were harvested (e.g., risk factors would be similar to that of eating an egg and a glass of milk). This is an effective treatment with less toxicity than the currently available alternative medicines.

In some embodiments, IgY antibodies can be useful against emergent or undifferentiated pathogen strains with a new combination of features, some of which already reside in other microbes. For example, new combinations of pathogen features result from random mutation, inclusion of DNA from other microbes, or antibiotic-mediated selective evolution. These events create new, highly virulent pathogen strains with limited response to existing medical treatments.

In this embodiment, antibodies can be designed to bind to several closely related epitopes that are present within a structurally related cluster of antigens. These antigens may differ markedly in other respects, and may originate from diverse sources, organisms, or species. An inoculant or immunogen can be selected to be a common or preserved component or region of the targeted antigen cluster, while ignoring the variable or distinguishing components or regions of the individual members of the cluster of related antigens. The method involves the preparation of a vaccine which will be administered to animals and which comprises an appropriate immunogen with characteristics that elicit the production of antibodies that are cross-reactive to desired instances of that epitope, but which are not reactive to other epitopes.

One example of this embodiment includes the production of antitoxin antibodies that are specifically reactive to clusters of structurally related toxins. These example antibodies would have effect without regard to the species originating the toxin. For example, the antibodies raised against the structurally related toxins may be neutralizing antibodies, capable of neutralizing or inactivating the biological activity of the target toxins.

This method can be extended to include any number of toxin clusters, and to include broad-spectrum neutralizing antibodies against mediators of other toxin-like reactions (for example viral toxin-like phenomena). Using these antibodies to prepare a therapeutic as disclosed herein, symptoms and pathology may be managed or prevented without knowledge of the infectious causes, or in cases where there are multiple infectious causes.

Protocols for immunizing the hens of the avian species with the vaccine may be according to those known in the art for initiating antibody production. In an example, the hens may receive two or more vaccinations at least two weeks apart. In some embodiments, the vaccinations may begin when the hens are 18 weeks of age or older. Booster vaccines may be given to the hens 6 months after the first vaccination.

In some embodiments, the vaccines are administered to the hens of the avian species subcutaneously. In other embodiments, the vaccines are administered through intramuscular, oral, intravenous, nasal, spray, or dermal procedures.

After the immunization process, whole shell eggs may be collected from the hens of the avian species. These eggs contain concentrated IgY which bind to the one or more pathogens against which the laying chicken was vaccinated. In other embodiments, the yolk of the eggs may be isolated from the egg whites.

A dehydrated egg powder may be produced from the eggs (either whole shell or isolated yolks) according to procedures known in the art. The drying temperature may be at least 138 degrees F., but not to more than 150 degrees F. which is sufficient to pasteurize the egg and dry to powder within 15 hours. The dehydrated egg product may then be ground to produce a powder suitable for mixing.

In another embodiment, the eggs can be spray dried. In this embodiment, the liquid eggs may be pasteurized at 140 degrees F. immediately prior to spray dry using dedicated food quality process equipment following relevant regulatory guidelines. In some embodiments, high pressure pasteurization can be used. The dried egg product may then be ground produce a powder suitable for mixing.

Additionally, other drying processes, lyophilization, pasteurization, and preservation methods may be used to process the eggs. Furthermore, the antibodies in the eggs may be concentrated, separated, or purified in various ways known in the art. The antibodies produced as disclosed herein may be purified, treated, or retained in the egg material for use in manufacturing the disclosed therapeutic or prophylactic treatment.

In some embodiments, other antibody types, antibody fragments, recombinant antibodies, monoclonal antibodies, polyclonal antibodies, or various antibody mixtures derived from bioreactor or non-avian animal sources can be combined with IgY antibodies. Such antibodies can include IgA, IgM, or other antibodies.

In some embodiments, the colostrum used to protect the IgY antibodies may be bovine colostrum. In some embodiments, the colostrum may be collected from non-hyperimmune ruminants. In some embodiments, non-hyperimmune ruminants may be non-hyperimmune cattle. In some embodiments, the colostrum may comprise of whole colostrum. The colostrum may be dehydrated and ground to a powder using techniques known in the art. Methods described herein for dehydrating the egg preparation may also be used to prepare colostrum powder.

Colostrum serves to slows degradation of the mixture of IgY antibodies in animal gut and provide additional protective and efficacious attributes to the antibody preparation. Any combination of antibodies may be used within a colostrum matrix, including but not limited to a combination of anti-pathogen, anti-toxin, and anti-adhesin antibodies.

In addition to colostrum, other protein sources may be used as a protective material that slows degradation of the mixture of IgY antibodies in an animal gut and can be mixed with the egg powder preparation. While colostrum includes antibodies derived from the lactating animal, its purpose in this composition is to act as a carrier and to protect the IgY antibodies derived from the egg powder preparation. Examples of other protein sources for use in preparing the protective protein matrix include serum albumin, for example, bovine serum albumin. Dehydrated egg whites may also be used as a protein matrix. While liquid egg whites as found in a chicken egg have approximately 10% protein, a more concentrated protein mixture may be created by dehydrating the egg whites to produce a powder that is added to the egg powder preparation. Protein powder derived from other animal tissues, for example, muscle, gelatin, or collagen of non-hyperimmune animals may also be dehydrated and used to create a powdered protein matrix. Yeast, whey, or whole milk are additional examples of protein sources which may be dehydrated to create a powdered protein matrix.

In yet another embodiment, a protective material other than, or in addition to, colostrum or protein can be used. Such a protective material should slow or reduce degradation of the mixture of IgY antibodies in an animal gut. For example, a protective material can include polymeric material, a polysaccharide material, a fatty material, or their mixtures or combinations. For example, in some embodiments chitosan, alginates, lecithin, cholesterol, or gums can be used. These can be used to coat IgY containing material or be used in various encapsulation methods including but not limited to microcapsules or liposome encapsulation. In some embodiments, the encapsulating material can act to hold together the mixture of IgY antibodies and protective material, advantageously slowing digestive degradation.

In some embodiments, additives or additional substances can be combined with a mixture of IgY and IgA, IgM, or other antibodies for their protective, stabilizing, or other utilities. These can include but are not limited to chemical buffering agents such as sodium bicarbonate, magnesium carbonate, or calcium carbonate. Other pharmaceutical grade buffering agents made by partially neutralizing a weak acid can include trisodium citrate, sodium lactate, trisodium phosphate, or sodium acetate. Other additives can include thermal protection agents such as sucrose, maltose, glycerol, or glycine. In some embodiments, dyes or tracking agents can be added. Other additives, including preservatives or flavorings, may also be included in the final mixture.

In some embodiments one or more of electrolytes, vitamins, and one or more probiotic cultures may also be included in the therapeutic to further support treatment of diarrhea. Probiotics are microbes that are normally found in the gut. They may be bacteria or yeast. When present in proper amounts, probiotic microbes aid in digestion, inhibit growth of pathogenic organisms, and synthesize nutrients. They may also support the host's immune system or have anti-inflammatory activity. In fact, different probiotic strains provide different benefits to the host. It is for at least this reason that probiotic supplements are often provided as a mixture of multiple strains. The mixture may include a plurality of bacteria strains, a plurality of yeast strains, or a plurality of both bacteria and yeast strains. In their absence or in reduced amounts, pathogenic microbes may proliferate in the gut creating an opportunistic infection.

In other embodiments prebiotics can be used to provide nutrients for the probiotic microbes. The protein matrix may act as a prebiotic. Other prebiotics which may be included are fructooligosaccharides (FOS), beet pulp, raw garlic, dandelion greens, wheat dextrin, chicory, fermented vegetables, and other prebiotics known in the art.

In some embodiments, once the preparation of dried egg material including IgY antibodies and the protective material and/or additives are prepared, the two may be mixed to create a powdered substance for using in treating a mammalian species. The protective protein powder may be provided in proportions of between 1% to 80% by weight of the egg and protein mixture. The powdered egg mixture may be provided in proportions of between 80% and 1% by weight of the egg and protein mixture. In some embodiments, the mixture contains approximately 55% protein or other protective powder and approximately 45% powdered egg preparation by weight. In some embodiments the mixture contains approximately 45% protein or protective powder and approximately 55% powdered egg preparation by weight. In some embodiments, the mixture contains approximately 50% protein or protective powder and approximately 50% powdered egg preparation by weight.

In some embodiments a mixture of IgY and IgA, IgM, or other antibodies contained in a therapeutic egg powder preparation can be administered as an oral, nasal, or mucosal preparation. In other embodiment, the therapeutic egg powder preparation can be prepared for administration as a suppository, intravenous injection in a saline or other suitable solution, or through any other suitable method.

In one embodiment the therapeutic may be administered by sprinkling the dry product onto food which the mammalian species may then ingest. The therapeutic in dry form may be mixed with water or other ingestible liquid and mixed into or decanted onto food which the mammalian species may then ingest. The therapeutic in dry form may be mixed with water or other ingestible liquid and administered into the mammalian species mouth using a syringe or provided for the mammalian species to drink or administered directly into the mammalian species stomach through a nasogastric tube. In some embodiments, as will be understood, the combined IgY, protective material, and optional additives can be prepared as a solid, gel, liquid, or aerosol formulation. Such therapeutic or prophylactics can be processed to produce tablets, chewable pills, syrups, elixirs, or aqueous suspensions. Any form known in the art which may be administered to an animal is within the scope of this disclosure. In summary, any method of administering the product into the bird species to be treated is within the scope of this disclosure.

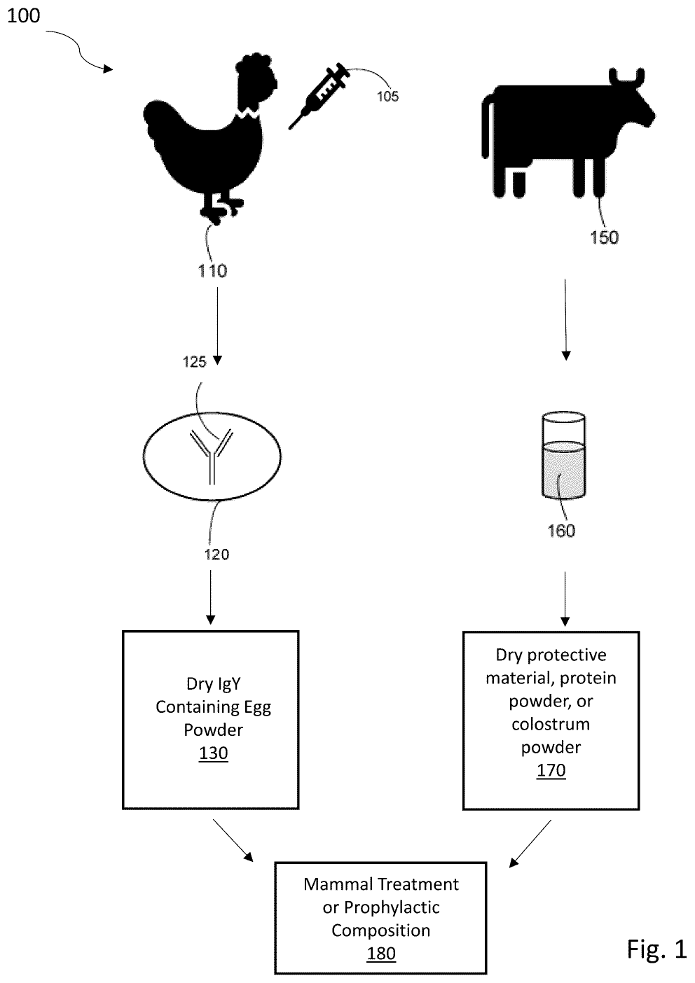

FIG. 1 describes an embodiment 100 of a preparation of the egg powder and protective material. In FIG. 1, chicken 110 is an avian species selected to receive vaccine 105 which includes multiple antigens associated with pathogens or toxins. Chicken 110 then lays egg 120 which contains antibodies, including IgY antibody 125. Many antibodies are present in egg 120 although only IgY antibody 125 is depicted for purposes of clarity. Egg 120 is converted to a dry egg powder using techniques described herein. In one embodiment, the protective material can be a protein matrix prepared from non-hyperimmune bovine colostrum. Lactating cow 150 is one example of a non-hyperimmune animal from which colostrum 160 is collected. Colostrum 160 is converted to a dry colostrum powder 170 using techniques described herein. Dry egg powder 130 and dry colostrum powder 170 are combined in ratios disclosed herein to produce therapeutic or prophylactic composition 180.

The egg and colostrum (or other matrix protein) mixture may be provided in powdered form. Alternatively, the egg and colostrum mixture may be processed to produce tablets, chewable pills, syrups, elixirs, or aqueous suspensions. Any form known in the art which may be administered orally to an animal is within the scope of this disclosure. Other additives, including preservatives or flavorings, may be included in the final mixture.

The therapeutic may be provided to a mammalian species in doses that may depend on the animal's body weight, the severity of the disease, and whether the therapeutic is being used to treat existing illnesses or prophylactically. In an example, a single dose may comprise 3-10 grams of the powdered egg and protein matrix mixture, excluding other additives which may be present in the final product. In some embodiments, a single dose may comprise approximately 5 grams of the powdered egg and protein matrix mixture, excluding other additives which may be present in the final product.

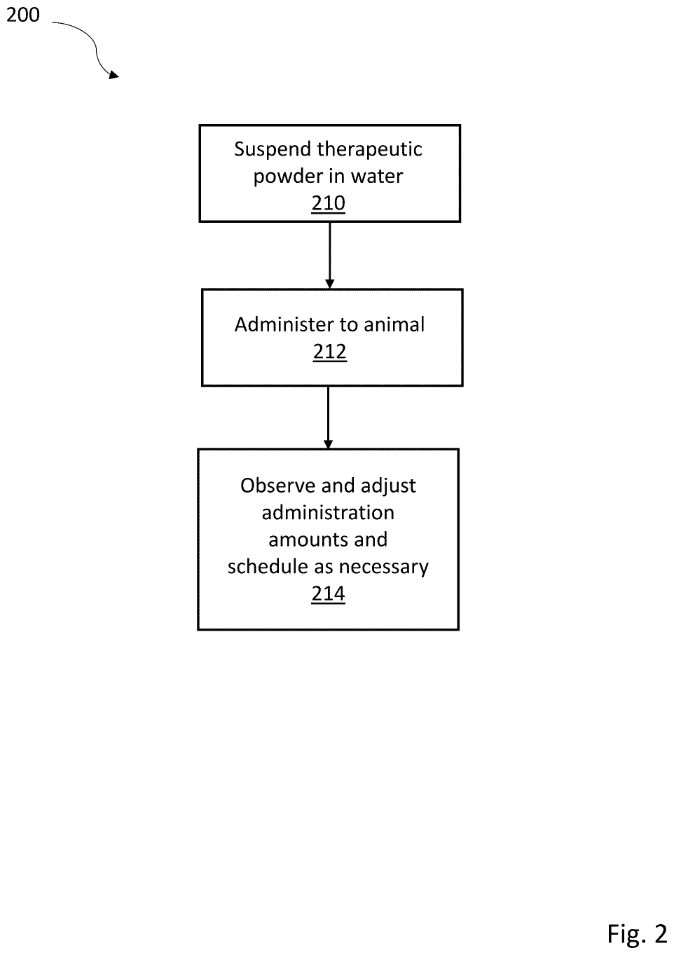

FIG. 2 summarizes an embodiment 200 in which an animal is treated with the disclosed composition. In step 210, a user suspends 5 g of the powdered therapeutic as described herein in 2 ounces of water. The suspended therapeutic is administered orally to an animal suffering from a pathogen or toxin (step 212). The animal is then observed for improvement in symptoms and the administration amounts and schedule are adjusted as necessary (step 214).

It is to be understood that the embodiments of the invention herein described are merely illustrative of the application of the principles of the invention. Reference herein to details of the illustrated embodiments is not intended to limit the scope of the claims, which themselves recite those features regarded as essential to the invention. The following examples are intended to illustrate but not limit the invention.

### Example 1—Preparation of IgY Containing Therapeutic for Mammal Species Treatment

An avian species (chicken breed Rhode Island Red) were housed, fed, and cared for according to standard protocol for commercial egg-laying hens. They were raised from hatchlings and fed a high protein diet comprising 20% or more protein for the first 10-15 weeks after hatching. Feed included 2.5-5.0 g calcium per day. Afterwards, the hens were fed a diet comprising less than 20% protein. Overall, the range of protein in the chicken feed was between 14-22 g per day.

The hens were vaccinated by subcutaneous injection with four commercially available animal vaccines. The first was ScourGuard 4KC available from Zoetis (Parsippany, New Jersey). This vaccine was a designed to prevent diarrhea caused by bovine rotavirus (serotypes G6 and G10), bovine coronavirus, enterotoxigenic strains of Escherichia coli having the K99 pili adherence factor, and Clostridium perfringens type C. The vaccine was provided as a liquid preparation of inactivated bovine rotavirus (serotypes G6 and G10) and coronavirus propagated on established cell lines, a K99 E. colibacterin, and C. perfringens type C toxoid and included an adjuvant to enhance the immune response. The second vaccine C. perfringens Type A Toxoid supplied by Elanco (Greenfield, Indiana) and included C. perfringens Type A, enterotoxin and an adjuvant. This vaccine was designed to prevent disease caused by the alpha toxin (Type A) of C. perfringens. The third vaccine was Campylobacter fetus-Jejuni Bacterin supplied by Colorado Serum (Denver, Colorado). This vaccine is an aqueous suspension of inactivated cultures (killed bacteria) of Campylobacter fetus and Campylobacter jejuni. It contained aluminum hydroxide as an adjuvant and thimerosal as a preservative. The fourth vaccine was Neopar supplied by NeoTech (Dresden, Tennessee). This vaccine was a modified live virus vaccine containing a high antigenic mass per dose of a highly immunogenic strain of canine parvovirus and included gentamicin and amphotericin B as preservatives. Consequently, the hens were vaccinated against the following diarrhea-causing organisms: coronavirus, C. perfringens, rotavirus, Salmonella, E. coli, Campylobacter, and parvovirus.

Following vaccinations, including but not limited to those described with respect to Example 1, eggs were collected from the hens. A dehydrated egg powder was prepared from the whole shell eggs by drying the eggs as described herein and grinding the dehydrated product to produce an egg powder containing IgY. The IgY containing egg powder was combined in a ratio of 50:50 by weight with commercially available bovine colostrum powder which was derived from non-hyperimmune cattle. The mixture was stored at room temperature until reconstituted with water for use as described below. Each dose comprised 5 g of the mixture along with a vanilla flavoring for palatability.

### Example 2—Treatment of Parvovirus in 6-Week Old Canine Mammal

Canine parvovirus is a highly contagious viral disease that can produce a life-threatening illness. The virus attacks rapidly dividing cells in a dog's body, most severely affecting the intestinal tract. Parvovirus also attacks the white blood cells, and when young animals are infected, the virus can damage the heart muscle and cause lifelong cardiac problems.

There are few drugs or treatments generally available that can kill parvovirus. Standard of care treatment consists of aggressive supportive care to control symptoms. Dogs infected with parvovirus need intensive treatment in a veterinary hospital, where they receive antibiotics, drugs to control the vomiting, intravenous fluids and other supportive therapies. This entails considerable expense; the average hospital stay is 5 to 7 days at a cost of several thousand dollars. Even with medical support canine parvovirus mortality rates range from 5%-20%. Without treatment, the mortality rate is 90%.

A six-week-old puppy (canine) was stricken with severe diarrhea and taken to a veterinary hospital for diagnosis and treatment. The attending veterinarian diagnosed the patient with parvovirus infection, which was confirmed with an ELISA fecal test. With the pet owner's permission, in addition to standard of care treatment, the attending veterinarian administered an antibody treatment consisting of hyperimmunized egg powder (raised in vaccinated chickens as described herein) containing parvovirus antibodies mixed with whole colostrum powder. The formulation was mixed with a small quantity of water and administered orally to the patient. The next morning the patient was reexamined at the hospital by the veterinarian. The puppy was symptom free, with normal temperature, solid stool, and no evidence of physical distress. A fecal ELISA test confirmed the absence of parvovirus infection. The antibody/colostrum formulation effectively conferred passive immunity to the patient and eradicated the parvovirus infection and sequelae within 24 hours of treatment.

### Example 3—Treatment of Parvovirus in Study Group of 10 Canines

The objective of this study was to evaluate the tolerability and efficacy of the disclosed treatment in parvovirus induced diarrhea episodes for dogs residing in the United States. The study was an open-label, multi-center, study of canine patients between one month and five years of age. The selection and recruitment of patients for the trial was conducted by the supervising veterinarian or health provider. Additional clinical or animal shelter personnel assisted in subject care and logistics as necessary. Pet owner informed consent was obtained upon enrollment. All participating subjects presented with a “moderate” to “severe” diarrhea profile, as reported by the attending veterinarian. Laboratory diagnostic tests were performed to confirm that parvovirus was the causative agent or etiology of the canine diarrhea.

The study group consisted of ten parvovirus positive subjects, with all ten completing the study. All study subjects were observed, and data was collected, over 24 hours. Antibiotic and active hydration treatment was withheld for all subjects pending subject status review the following day.

The treatment was as disclosed herein and included hyperimmunized egg powder created from eggs laid by hens which had been immunized as described herein and whole colostrum powder created from bovine colostrum. The animals which provided the colostrum were not vaccinated with parvovirus vaccine. The treatment was packaged in single dose sachets containing 5 g of powder. The treatment was administered orally, with the contents of one sachet resuspended in approximately 2 oz. of standard drinking water. Test subjects were required to drink the entire suspension in one sitting, immediately after preparation was complete. Subjects remained under on-site observation for 10 minutes after administration.

Reported parameters as measured for each subject include stool frequency, stool consistency, and doctor reported well-being. These three criteria were found to be the most significant to both the attending veterinarians and the subject's owner in assessing the state and the improvement of the subject's condition.

Subjects were evaluated against standard of care states: bloody diarrhea; lethargy; anorexia; fever; vomiting; and severe weight loss. All subjects were rated as moderate to severely ill, up to and including states such as listless and minimally responsive. Dramatic improvements were observed in most subjects within 12 hours of initial administration of the disclosed therapy. Stool frequency and consistency returned to normal. Vomiting and bloody stool were eliminated. Normal activity resumed and no adverse side effects were observed. One test subject suffering declining well-being was treated with the disclosed therapy after undergoing several days of standard of care treatment. Although diarrhea-related symptoms subsided, the subject subsequently died from systemic failure believed to result from the initial parvovirus infection.

This study provides evidence that the disclosed treatment is highly effective in managing acute parvovirus diarrhea in canine patients with significant reductions in both the duration and severity of illness. It is also effective in the dietary management of acute parvovirus diarrhea, greatly reducing the length and severity of illness. The treatment is a safe, non-antibiotic dietary intervention that was well tolerated by the test subjects.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A composition for therapeutic or prophylactic treatment of mammals, the composition comprising:
a mixture of IgY antibodies derived from one or more eggs laid by one or more avian species, wherein each of the one or more avian species have been vaccinated with one or more of the plurality of antigens; and
a protective material that slows degradation of the mixture of IgY antibodies in an animal gut; and
an encapsulating material to hold together the mixture of IgY antibodies and the protective material.

2. The composition of claim 1, wherein the avian species comprises at least one of a chicken, duck, turkey, goose, and ostrich.

3. The composition of claim 1, wherein the plurality of antigens are selected from the group consisting of: a species of the genus Clostridium, a species of the genus Yersinia, a species of the genus Brachyspira, a species of the genus Campylobacter, Escherichia coli, Helicobacter spp., a species of the genus Salmonella, a species of the genus Leptospira, Providencia alcalifaciens, Mycobacterium paratuberculosis, Lawsonia intracellularis, bovine viral diarrhea virus, infectious bovine rhinotracheitis virus, coronavirus, rotavirus, parvovirus, paramyxovirus, hepatitis virus, feline leukemia virus, feline immunodeficiency virus, feline panleukopenia virus, astrovirus, transmissible gastroenteritis, Giardia, Coccidia, Entamoeba histolytica, Cryptosporidium, roundworm, whipworm, spirochetes, yeasts, and molds.

4. The composition of claim 1, wherein the protective material is colostrum is derived from a ruminant.

5. The composition of claim 1, wherein the protective material comprises at least 1 percent of the composition by weight.

6. The composition of claim 1, wherein the protective material further comprises an encapsulating material.

7. The composition of claim 1, wherein the protective material further comprises a polymeric material.

8. The composition of claim 1, wherein the protective material further comprises a polysaccharide material.

9. The composition of claim 1, wherein the protective material further comprises a fatty material.

10. An intravenously deliverable composition for therapeutic or prophylactic treatment of animals, the composition comprising:
a mixture of IgY antibodies derived from one or more eggs laid by one or more avian species, wherein each of the one or more avian species have been vaccinated with one or more of the plurality of antigens;
a mixture of at least one of IgA and IgM antibodies; and
a protective material that slows degradation of the mixture of IgY with the mixture of one of IgA or IgM antibodies and is soluble in saline solution.

11. The composition of claim 10, wherein the avian species comprises at least one of a chicken, duck, turkey, goose, and ostrich.

12. The composition of claim 10, wherein the plurality of antigens are selected from the group consisting of: a species of the genus Clostridium, a species of the genus Yersinia, a species of the genus Brachyspira, a species of the genus Campylobacter, Escherichia coli, Helicobacter spp., a species of the genus Salmonella, a species of the genus Leptospira, Providencia alcalifaciens, Mycobacterium paratuberculosis, Lawsonia intracellularis, bovine viral diarrhea virus, infectious bovine rhinotracheitis virus, coronavirus, rotavirus, parvovirus, paramyxovirus, hepatitis virus, feline leukemia virus, feline immunodeficiency virus, feline panleukopenia virus, astrovirus, transmissible gastroenteritis, Giardia, Coccidia, Entamoeba histolytica, Cryptosporidium, roundworm, whipworm, spirochetes, yeasts, and molds.

13. The composition of claim 10, wherein the protective material is colostrum derived from a ruminant.

14. The composition of claim 10, wherein the protective material comprises at least 1 percent of the composition by weight.

15. The composition of claim 10, wherein the protective material further comprises an encapsulating material.

16. The composition of claim 10, wherein the protective material further comprises a polymeric material.

17. The composition of claim 10, wherein the protective material further comprises a polysaccharide material.

18. The composition of claim 10, wherein the protective material further comprises a fatty material.
